(19) United States
(12) Patent Application Publication
(10) Pub. No.: US 2025/0200838 A1
(43) Pub. Date: Jun. 19, 2025

(54) RECONSTRUCTING METHOD AND APPARATUS OF MEDICAL IMAGE, COMPUTER DEVICE, AND STORAGE MEDIUM

(71) Applicant: Shanghai United Imaging Healthcare Co., Ltd., Shanghai (CN)

(72) Inventors: Xu Liu, Shanghai (CN); Youjun Sun, Shanghai (CN)

(21) Appl. No.: 18/986,916

(22) Filed: Dec. 19, 2024

(30) Foreign Application Priority Data
Dec. 19, 2023 (CN) 202311754545.7

(51) Int. Cl. G06T 11/00

(52) U.S. Cl. CPC G06T 11/005; G06T 2210/41

(57) ABSTRACT

A reconstructing method and a reconstructing apparatus of a medical image, a computer device, and a storage medium are provided. The method includes: acquiring a signal data set obtained by performing signal detection on a detected object via a detector array, determining an effective detecting range of each detector in the detector array when a detecting scenario is a target scenario, and extracting target signal data of each detector within a respective effective detecting range from the signal data set, and reconstructing a medical detecting image of the detected object based on the target signal data. The detector array is configured to siege the detected object in three dimensions and detect a trace signal emitted by the detected object to obtain the signal data set.

### CROSS-REFERENCE TO RELATED APPLICATION

The present disclosure claims priority to Chinese patent applications No. 202311754545.7,filed on Dec. 19, 2023, titled “RECONSTRUCTING METHOD AND APPARATUS OF MEDICAL IMAGE, COMPUTER DEVICE, AND STORAGE MEDIUM”, the content of which is hereby incorporated by reference in its entirety.

### TECHNICAL FIELD

The present disclosure generally relates to the field of medical imaging, and in particular, to a reconstructing method of a medical image, a reconstructing apparatus of a medical image, a computer device, a storage medium, and a computer program product.

### BACKGROUND

A positron emission tomography (PET) is a medical imaging technology used to observe biological activities and functions inside a detecting object. A radioactive tracer (e.g. glucose labeled by a radioactive isotope) is injected to a body of the detecting object, and then a PET scanner detects and records a spatial distribution of the radioactive tracer in the body.

During a PET detection, trace signals that are emitted by the radioactive tracer (such as two gamma ray signals that moves along opposite directions are produced by annihilation of a positive electron and a negatron when the positive electron meet with the negatron in the body) are detected and recorded by the PET scanner. A tomographic image of the body is generated by analyzing and reconstructing data of the trace signals, to observe and evaluate metabolic activity, blood flow, brain function of different tissues and organs.

However, in a current PET system, a data volume and a counting rate are multiplied due to a system sensitivity increase. As a result, in a corresponding special scenario (a high-active radiation source scenario such as myocardial injection and dynamic scanning in clinical practice), the PET system captures a large quantity of trace signals in a short time. These trace signals bring great pressure to data processing, data storage, and reconstruction of the PET system, and affect data availability, correctness, and reconstructing image consistency.

### SUMMARY

According to various embodiments of the present disclosure, a reconstructing method of a medical image, a reconstructing apparatus of a medical image, a computer device, a storage medium, and a computer program product are provided.

In a first aspect, a reconstructing method of a medical image is provided, including: acquiring a signal data set obtained by performing signal detection on a detected object via a detector array, determining an effective detecting range of each detector in the detector array when a detecting scenario is a target scenario, and extracting target signal data of each detector within a respective effective detecting range from the signal data set, and reconstructing a medical detecting image of the detected object based on the target signal data. The detector array is configured to siege the detected object in three dimensions and detect a trace signal emitted by the detected object to obtain the signal data set.

In an embodiment, determining an effective detecting range of each detector in the detector array further includes: acquiring a detecting region of the detector array for the detected object, and determining, based on the detecting region, the effective detecting range corresponding to each detector.

In an embodiment, the detecting region includes a first projection region of the detected object in an axial view of the detector array. Determining, based on the detecting region, the effective detecting range corresponding to each detector further includes: determining, based on the first projection region, a first effective detecting range of each detector in the axial view.

In an embodiment, determining the effective detecting range corresponding to each detector further includes: for the axial view, taking the detector as an endpoint and two corresponding first siege lines as sidelines, sieging the first projection region to obtain a first siege region, and taking a view range corresponding to the first siege region as the first effective detecting range. An end of each of the first siege lines is located at the endpoint, the other end of each of the first siege lines is located within an axial length range of the detector array, and the first siege region is configured to siege at least a part of the first projection region.

In an embodiment, the detecting region includes a second projection region of the detected object in a circumferential view of the detector array. Determining, based on the detecting region, the effective detecting range corresponding to each detector further includes: determining, based on the second projection region, a second effective detecting range of each detector in the circumferential view.

In an embodiment, determining the effective detecting range corresponding to each detector further includes: for the circumferential view, taking the detector as an endpoint and two corresponding second siege lines as sidelines, sieging the second projection region to obtain a second siege region, and taking a view range corresponding to the second siege region as the second effective detecting range. An end of each of the second siege lines is located at the endpoint, the other end of each of the second siege lines is located within a circumferential length range of the detector array, and the second siege region is configured to siege all the second projection region.

In an embodiment, each detector has at least one coincidence line within the respective effective detecting range, an end of the at least one coincidence line is a local-end detector corresponding to the effective detecting range, and the other end of the at least one coincidence line is a peer-end detector. Extracting target signal data of each detector within the respective effective detecting range from the signal data set further includes: extracting, from the signal data set, target trace signals of the local-end detector and the peer-end detector those are on each of the at least one coincidence line, and taking the target trace signals as the target signal data. Each of the at least one coincidence line belongs to the corresponding effective detecting range of each detector.

In an embodiment, determining the detecting scenario of the detector array for the detected object based on the data feature of the signal data set further includes: performing signal identification on each signal data in the signal data set, determining statistical data of the trace signal emitted from the detected object based on the radioactive tracer, and determining the detecting scenario of the detector array based on an activity level. The statistical data is configured to represent the activity level of the radioactive tracer in the detected object.

In an embodiment, performing signal identification on each signal data in the signal data set, and determining statistical data of the trace signal sent from the detected object based on the radioactive tracer further includes: identifying a preset radioactive signal and a positron signal that are emitted by the detected object in a coincident time window, obtaining a data amount of the radioactive signal and a counting rate of the positron signal, and taking either or both of the data amount and the counting rate as the statistical data for the trace signal.

In a second aspect, a reconstructing apparatus of a medical image is further provided in the present disclosure, including: means for acquiring a signal data set obtained by performing signal detection on a detected object via a detector array, means for determining an effective detecting range of each detector in the detector array when the detecting scenario is a target scenario, and means for extracting target signal data of each detector within a respective effective detecting range from the signal data set, and reconstructing a medical detecting image of the detected object based on the target signal data. The detector array is configured to siege the detected object in three dimensions and detect a trace signal emitted by the detected object to obtain the signal data set.

In a third aspect, a computer device is further provided in the present disclosure, including a memory and a processor. The memory is configured to store an executable instruction of the processor, and the processor is configured to execute the executable instruction to implement the reconstructing method of the medical image.

In a fourth aspect, a computer-readable storage medium is further provided in the present disclosure, including program data. When the program data is executed by a processor of a computer device, the compute device can implement the reconstructing method of the medical image.

In a fifth aspect, a computer program product is further provided in the present disclosure, including a computer program. The computer program is executed by a processor to implement the reconstructing method of the medical image.

Details of one or more embodiments of the present disclosure are set forth in the following accompanying drawings and description. Other features, objectives, and advantages of the present disclosure become obvious with reference to the specification, the accompanying drawings, and the claims.

### BRIEF DESCRIPTION OF THE DRAWINGS

In order to more clearly illustrate the technical solutions in the embodiments of the present disclosure or the related technology, the accompanying drawings to be used in the description of the embodiments or the related technology will be briefly introduced below, and it will be obvious that the accompanying drawings in the following description are only some of the embodiments of the present disclosure, and that, for one skilled in the art, other accompanying drawings can be obtained based on these accompanying drawings without putting in creative labor.

FIG. 1 is a schematic diagram of an application environment of a reconstructing method of a medical image in an embodiment.

FIG. 2 is a flowchart of a reconstructing method of a medical image in an embodiment.

FIG. 3 is a schematic diagram of a detector array in an embodiment.

FIG. 4 is a schematic diagram of a coincidence line of a detector in an embodiment.

FIG. 5 is a flowchart of determining an effective detecting range of a detector in an embodiment.

FIG. 6 is a schematic diagram of an effective detecting range of a detector in an axial view in an embodiment.

FIG. 7 is a schematic diagram of an effective detecting range of a detector in a circumferential view in an embodiment.

FIG. 8 is a block diagram of a PET/CT system in an embodiment.

FIG. 9 is a block diagram of a reconstructing apparatus of a medical image in an embodiment.

FIG. 10 is a block diagram of a reconstructing apparatus of a medical image in an embodiment.

FIG. 11 is a block diagram of a computer for reconstructing a medical image in an embodiment.

FIG. 12 is a block diagram of a computer-readable storage medium for reconstructing a medical image in an embodiment.

FIG. 13 is a block diagram of a computer program product for reconstructing a medical image in an embodiment.

### DETAILED DESCRIPTION OF THE EMBODIMENT

To make objectives, technical solutions, and advantages of the present disclosure clearer, the following further describes the present disclosure in detail with reference to the accompanying drawings and embodiments. It should be understood that the specific embodiments described herein are merely used to explain the present disclosure, and are not intended to limit the present disclosure.

The term “and/or” in the embodiments of the present disclosure refers to any and all possible combinations including one or more of associated enumerated items. It is also noted that when used in this specification, “include/comprise” specifies presence of stated features, integers, steps, operations, elements and/or components, but not rule out presence or addition of one or more other features, integers, steps, operations, elements and/or components and/or groups thereof.

The terms “first” and “second” in the present disclosure are used to distinguish between different objects, and are not used to describe a specific sequence. In addition, the terms “include”, “have” and any variations thereof are intended to cover the inclusion of non-exclusive. For example, a process, a method, a system, a product, or a device that includes a series of steps or units is not limited to the listed steps or units, but alternatively further includes an unlisted step or unit, or alternatively further includes another step or unit inherent to the process, the method, the product, or the device.

In addition, in the present disclosure, although the terms “first”, “second” and the like are repeatedly used to describe various operations (elements, applications, instructions, or data) and the like, these operations (elements, applications, instructions, or data) are not limited to these terms. These terms are used only to distinguish one operation (an element, an application, an instruction, or data) from another operation (an element, an application, an instruction, or data).

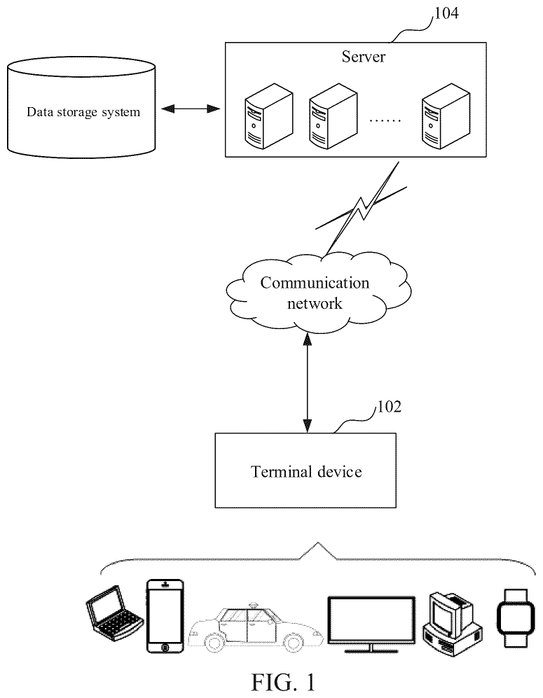

A reconstructing method of a medical image provided in an embodiment of the present disclosure is applied to an application environment shown in FIG. 1. A terminal 102 is in communication with a server 104 by a communication network. A data storage system may store data that needs to be processed by the server 104. The data storage system may be integrated on the server 104, or may be placed on a cloud or a network server.

In some embodiments, referring to FIG. 1, firstly, the server 104 acquires a signal data set obtained by performing signal detection on a detected object via a detector array. The detector array is configured to siege the detected object in three dimensions and detect a trace signal emitted by the detected object to obtain the signal data set. When a detecting scenario is a target scenario, the server determines an effective detecting range of each detector in the detector array. Finally, the server 104 extracts target signal data of each detector within a respective effective detecting range from the signal data set, and reconstructs a medical detecting image of the detected object based on the target signal data.

In some embodiments, the terminal 102 (such as a mobile terminal and a fixed terminal) may be implemented in various forms. The terminal 102 may be the mobile terminal that can reconstruct the medical detecting image of the detected object according to target signal data of the detector within the effective detecting range, such as a mobile phone, a smart phone, a notebook computer, a portable handheld device, a Personal Digital Assistant (PDA), a tablet computer (PAD), or the like. The terminal 102 may also be the fixed terminal that can reconstruct the medical detecting image of the detected object according to target signal data of the detector within the effective detecting range, such as an Automated Teller Machine (ATM), an automatic integrated computer, a digital TV, a desktop computer, a solid-state computer, or the like.

The following assumes that the terminal 102 is a fixed terminal. However, one skilled in the art will understand that, when there is an operation or a component specifically used for a mobile purpose, the reconstruction according to implementation manners disclosed in the present disclosure may also be applied to a terminal 102 with a mobile type.

In some embodiments, a data processing component running on the server 104 may load any of various additional server applications and/or intermediate layer applications that is being executed, such as HTTP (Hyper Text Transfer Protocol), FTP (File Transfer Protocol), CGI (Common Gateway Interface), RDBMS (Relational Database Management System), or the like.

In some embodiments, the server 104 may implement a corresponding network function

by deploying a server (such as an independent server or a server cluster consisting of a plurality of servers). The server 104 may also be adapted to run one or more application services or software components of the terminal 102 described in the foregoing disclosure.

In some embodiments, the application services may include providing a user with a service interface (a trace signal collection interface, a display interface of signal data processing, or the like) of reconstructing the medical detecting image, a corresponding program service, or the like. The software components may include, for example, a Software Development Kit (SDK) or a client (APP) that has an image reconstructing function according to a detecting scenario.

In some embodiments, the SDK or the APP that has the image reconstructing function according to the detecting scenario and is provided by the server 104 may include a portal port that provides a one-to-one application service to a user in a foreground and multiple service systems that are located in a background to perform data processing, so as to extend an image reconstruction processing function application to the APP or the client, so that the user can use and access the image reconstruction processing function anywhere at any time.

In some embodiments, the image reconstruction processing function in the APP or the

client may be a computer program running in a user mode to complete a particular task or tasks that may interact with the user and have a visual user interface. The APP or the client may include two parts: A graphical user interface (GUI) and an engine (engine). The graphical user interface (GUI) and the engine can be used to provide multiple application services to the user in a form of a user interface.

In some embodiments, the user may input corresponding code data or control parameters to the APP or the client by using a preset input apparatus or an automatic control program, so as to execute the application service of the computer program in the server 104 and display the application service in the user interface.

In some embodiments, the operating system running on the APP or the client may include various versions of Microsoft Windows®, Apple Macintosh®, and/or Linux operating systems, various commercial or UNIX®-like operating systems (including but not limited to various GNU/Linux operating systems, Google ChromeROS, and the like), and/or a mobile operating system such as iOS®, Windows®Phone, Android®OS, BlackBerry®OS, and Palm®OS operating systems, and other online operating systems or offline operating systems, which are not specifically limited herein.

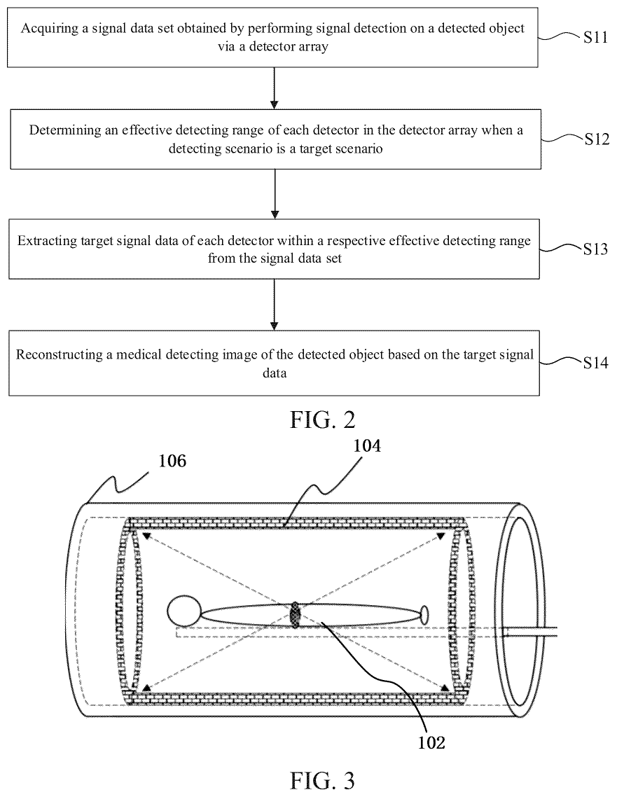

In some embodiments, referring to FIG. 2, a reconstructing method of a medical image is provided, the method is applied to the server 104 in FIG. 1 as an example for description, and the method includes following step 11 to step 14.

Step 11 includes that acquiring a signal data set obtained by performing signal detection on a detected object via a detector array.

The detected object may be a medical diagnosis and treatment object that has been injected with a radioactive tracer, such as a human object, an animal object, a plant object, or the like. The detected object may be a detected module.

In an embodiment, the radioactive tracer is a radioactive isotope-labeled compound that acts in a living object and can participate in blood flow or metabolism of a tissue of the object, including radioactive isotope-labeled glucose or the like.

In an embodiment, the detector array is configured to siege the detected object in three dimensions and detect a trace signal emitted by the detected object to obtain the signal data set.

In some embodiments, the radioactive tracer emits positrons after being injected into the detected object, and these positrons are annihilated when meeting negatrons in the detected object, to produce two gamma rays with relative motion, and then detectors in the detector array detect and record the gamma rays to obtain the signal data set.

In an exemplary embodiment, referring to FIG. 3, FIG. 3 is a schematic diagram of a detector array in an embodiment of the present disclosure. Referring to FIG. 3, the detector array 104 may include one or more detector units, and the one or more detector units may be encapsulated to form a cylindrical three-dimensional detector box, i.e., the detector array 104 may be arranged in a circular configuration (also referred to as a detecting ring) in a cross-section. The detector array 104 may be covered and protected by a housing 106, and the housing 106 may be a hollow cylinder. A region sieged by the detector array 104 may be a detecting region, and a to-be-scanned detected object 102 may be accommodated in the detecting region, so as to siege the detected object 102 in three dimensions. When the detected object 102 is in a field of view of the detector array 104, the trace signal (e.g., a gamma photon signal, a positron signal, etc.) emitted from the detected object 102 may be detected by the detector array 104 to obtain the signal data set.

In some embodiments, the detector array 104 may include one or more crystal elements (e.g., detector crystals). When the trace signal (e.g., a gamma ray photon) reaches a detector crystal, the detector crystal may absorb energy of the trace signal, and convert absorbed energy into visible light. In some embodiments, the detector crystals may be arranged in N rows and M columns. N may be an integer greater than 0, and M may be an integer greater than 0. In some embodiments, a barrier material (e.g., a reflective film, etc.) may be filled between adjacent two detector crystals of the detector array 104. The detector crystal may use one or more types of crystals, including, e.g., one or a combination of NaI (TI), BGO, LSO, YSO, GSO, LYSO, LaBr₃, LFS, LuAP, LuI₃, BaF₂, CeF, CsI (TI), CsI (Na), CaF₂ (Eu), CdWO4, and YAP.

Step 12 includes that determining an effective detecting range of each detector in the detector array when a detecting scenario is a target scenario.

For example, an effective detecting range corresponding to the detector in a detecting scenario with high activity (the activity level of the radioactive tracer in the detected object is relatively high) may be a first range. An effective detecting range corresponding to the detector in a detecting scenario with medium activity (the activity level of the radioactive tracer in the detected object is relatively normal) may be a second range. An effective detecting range corresponding to the detector in a detecting scenario with low activity (the activity level of the radioactive tracer in the detected object is relatively low) may be a third range. Effective detecting ranges corresponding to the detector in different detecting scenarios are different. The first range is greater than the second range, and the second range is greater than the third range. The target scenario may be the detecting scenario with high activity, such as myocardial injection and dynamic scanning in clinical practice. The target scenario may also be a long-axis scanning scenario.

In an embodiment, each detector may have at least one coincidence line within the respective effective detecting range. An end of the at least one coincidence line may be a local-end detector corresponding to the effective detecting range, and the other end of the at least one coincidence line may be a peer-end detector.

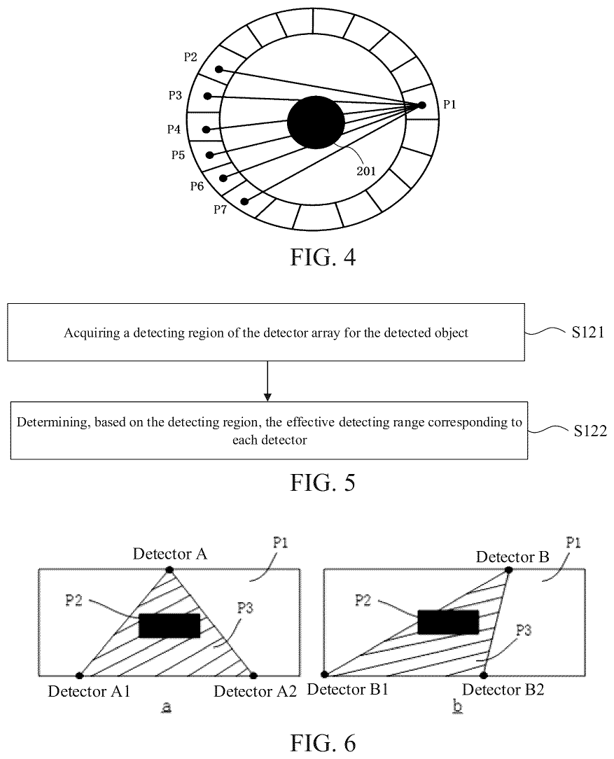

In an exemplary embodiment, referring to FIG. 4, FIG. 4 is a schematic diagram of a coincidence line of a detector in an embodiment of the present disclosure. Referring to FIG. 4, the schematic diagram is a schematic cross-sectional diagram of the detector array, and each detector may be arranged in a circular cylindrical manner, so as to siege the detected object 201 in three dimensions. A detector P1 may be a local-end detector, and a peer-end detector P2, a peer-end detector P3, a peer-end detector P4, a peer-end detector P5, a peer-end detector P6, and a peer-end detector P7 may be in a FOV (field of view) of the detector P1. A connection between the detector P1 and the peer detector P2 may be a coincidence line S1 (also referred to as a LOR (line of response)), a connection between the detector P1 and the peer detector P3 may be a coincidence line S2, a connection between the detector P1 and the peer detector P4 may be a coincidence line S3, a connection between the detector P1 and the peer detector P5 may be a coincidence line S4, and a connection between the detector P1 and the peer detector P6 may be a coincidence line S5, and a connection between the detector P1 and the peer detector P7 may be a coincidence line S6.

The method may further include that determining the detecting scenario of the detector array for the detected object based on a data feature of the signal data set.

Specifically, the server may first perform signal identification on signal data in the signal data set to determine statistical data of the trace signal emitted by the detected object based on the radioactive tracer. Then, the detecting scenario of the detector array may be determined based on the statistical data.

The statistical data is configured to represent an activity level of the radioactive tracer in the detected object. The higher the statistical data of the trace signal emitted by the detected object is, the higher the activity level of the radioactive tracer is, and the higher the activity level of the detecting scenario of the detector array is. The lower the statistical data of the trace signal emitted by the detected object is, the lower the activity level of the radioactive tracer is, and the lower the activity level of the detecting scenario of the detector array is.

In an embodiment, determining, by the server, the statistical data of the trace signal emitted by the detected object based on the radioactive tracer may further include: identifying a radioactive signal and a positron signal that are emitted by the detected object in a coincident time window, obtaining a data amount of the radioactive signal and a counting rate of the positron signal, and taking either or both of the data amount and the counting rate as the statistical data of the trace signal.

Exemplarily, the signal data set collected by the detector array in the coincident time window (e.g., 0 μs to 15 μs) may include X pieces of signal data. The server may identify the X pieces of signal data one by one to determine the data amount of the radioactive signal (e.g., gamma rays) and the counting rate of the positron signal, and then use either or both of the data amount and the counting rate as the statistical data of the trace signal.

In an embodiment, during a scanning process, reconstruction preview may be performed on a real-time data signal in slice time to obtain a brief outline of the detected object, so as to identify the detecting scenario.

In an embodiment, the detecting scenario may be determined according to a scanning protocol selected by the user.

Step 13 includes that extracting target signal data of each detector within a respective effective detecting range from the signal data set.

Specifically, the server may extract, from the signal data set, a target trace signal detected by a local-end detector and a peer-end detector on each coincidence line in a corresponding effective detecting range of each detector, and take the target trace signal as the target signal data.

The target trace signal may be a trace signal that is generated on a coincidence line and corresponding to a coincidence event.

In some embodiments, the server may first take trace signals detected by the local-end detector and the peer-end detector on each coincidence line as a signal data group, to obtain a set of signal data groups of coincidence lines. Then, coincidence event determining based on coincidence time window may be performed on the signal data groups in the set of signal data groups, respectively. When it is determined that a coincidence event is generated, the server may record response data (including information such as energy strength, response time, and response location when the coincidence event is generated) of the trace signal, and store the response data of the trace signal as the target signal data.

After the detected object is injected with the radioactive tracer, the positron emitted by decay of radionuclide is combined with the negatron in the tissue to annihilate, thereby generating two gamma photons with equal energy and opposite directions. Since the two gamma photons have different distances in the body of the detected object, the time to arrive at the two detectors is also different. When the two gamma photons between which a degree of 180° is formed are detected by the local-end detector and the peer-end detector on the coincidence line in a specified coincidence time window (e.g., 0 us to 15 μs), a coincidence event is formed.

Step 14 includes that reconstructing a medical detecting image of the detected object based on the target signal data.

Specifically, the server may sequentially perform data preprocessing, algorithm reconstruction processing, image post-processing, and image display processing on the target signal data to obtain a corresponding medical detecting image.

Data preprocessing is configured to preprocess the collected target signal data, including energy cutting, time correction, scattering correction, or the like. These preprocessing steps may facilitate improving quality and accuracy of the image.

Algorithm reconstruction processing is configured to convert the preprocessed data into a three-dimensional PET image. Common reconstruction algorithms may include an iterative algorithm, a filtering back projection algorithm, or the like. These algorithms may restore an image according to a characteristic and a sampling manner of the signal data by a mathematical model and an image processing technology.

Image post-processing is configured to perform post-processing on the reconstructed three-dimensional PET image, including noise removal, correction of non-uniformity of a scanner, correction of gradient drift, contrast enhancement, and smoothing of the image. These post-processing steps may facilitate improving visualization and diagnostic capabilities of the image.

Image display processing is configured to display the reconstructed PET image on a screen for analysis and diagnosis by a physician and a researcher.

In the above reconstructing process of the medical image, firstly, the signal data set obtained by performing signal detection on the detected object is acquired via the detector array. The detector array is configured to siege the detected object in three dimensions and detect the trace signal emitted by the detected object to obtain the signal data set. Then, the detecting scenario of the detector array for the detected object is determined based on the data feature of the signal data set. When the detecting scenario is the target scenario, the effective detecting range of each detector in the detector array is determined. Finally, the target signal data of each detector within the respective effective detecting range is extracted from the signal data set, and the medical detecting image of the detected object is reconstructed based on the target signal data. In this way, in one aspect, the detection scenario of the detector array for the detected object is determined by the data feature of the detected signal data set, and then the effective detecting range of each detector is determined according to the corresponding detection scenario, so as to reconstruct the medical detecting image based on the target signal data in the corresponding effective detecting range. Therefore, a procedure of reconstructing the medical image is optimized, and compared with a manner in the related art, reconstruction efficiency of the medical image is effectively improved by a standardized execution program, and human and material power consumption is reduced. In another aspect, the trace signal emitted by the detected object is first detected by sieging the detected object in three dimensions, and then the target signal data in the effective detecting range corresponding to each detector is extracted from the trace signal according to the detecting scenario of the detector array, so as to construct the medical detecting image. Therefore, accuracy and feasibility of reconstructing the medical image are improved, and it facilitates providing further effective reference information for subsequent medical treatment.

One skilled in the related art may understand that, in the foregoing method in a specific implementation manner, the disclosed method may be implemented in a more specific manner. For example, the foregoing described implementation in which the server determines the effective detecting range of each detector in the detector array according to the detecting scenario of the detector array for the detected object is merely exemplary.

In an exemplary embodiment, referring to FIG. 5, FIG. 5 is a flowchart of determining the effective detecting range of the detector in an embodiment of the present disclosure. At step 12, a process that determining the effective detecting range of each detector in the detector array when the detecting scenario is the target scenario may specifically execute technical content in the following manners.

Step 121 may include that acquiring a detecting region of the detector array for the detected object.

In an embodiment, the detecting region may be a physical space region of the detected object, which includes a first projection region of the detected object in an axial view of the detector array and a second projection region of the detected object in a circumferential view of the detector array.

Specifically, in the axial view of the detector array, a region that belongs to the detecting region of the detector array for the detected object and that is in an object contour region occupied by the detected object may be taken as the first projection region. In addition, in the circumferential view of the detector array, a region that belongs to the detecting region of the detector array for the detected object and that is in the object contour region occupied by the detected object may be taken as the second projection region. The axial view may be a field of view along a forward direction of a bed in the space inside the detector array. The circumferential view may be a field of view along a direction perpendicular to the forward direction of the bed in a cross-section of the space inside the detector array.

In an embodiment, the detecting region may be obtained based on a current scanning protocol of the detector array, a photographing region of a 2D/3D camera mounted in the detector array, or a concern region manually planned by a physician. A manner of determining the detecting region may be not specifically limited herein.

In some embodiments, the detector array may scan the detected object based on a preset scanning protocol. Detecting regions of the detector array for the detected object in different protocols may be different.

In some embodiments, the scan protocol may include a scan program and a parameter that are configured when the detector array scans and detects the detected object. The scan protocol may include a corresponding scan parameter, a scan time, and a scan region.

Exemplarily, when the current scanning protocol represents that the detector array performs

scanning of a head of a patient, the detecting region of the detector array may be a space region of the head of the patient, and the space region of the head may be is displayed in the axial view of the detector array as the first projection region of a head entity, and may be displayed in the circumferential view as the second projection region of the head entity. When the current scanning protocol represents that the detector array performs scanning of a heart of the patient, the detecting region of the detector array may be a space region of the heart of the patient, and the space region of the heart may be displayed in the axial view of the detector array as the first projection region of a cardiac entity, and may be displayed in the circumferential view as the second projection region of the cardiac entity.

Step 122 may include that determining, based on the detecting region, the effective detecting range corresponding to each detector.

In an embodiment, for the axial view, the server may determine a first effective detecting range of each detector in the axial view based on the first projection region

Specifically, the server may first siege the first projection region by using a detector as an endpoint and two corresponding first siege lines as sidelines, to obtain a first siege region. Then, a view range corresponding to the first siege region may be taken as the first effective detecting range.

An end of each of the first siege lines may be located at the endpoint, the other end of each of the first siege lines may be located within an axial length range of the detector array, and the first siege region is configured to siege at least a part of the first projection region.

In an exemplary embodiment, referring to FIG. 6, FIG. 6 is a schematic diagram of an effective detecting range of a detector in an axial view in an embodiment of the present disclosure. Referring to a and b in FIG. 6, a rectangular region PI may be a display region of the detector array in the axial view, and each detector in the detector array may be arranged at an edge position of the rectangular region P1, so as to perform signal detection in a first projection region P2 at which a radiation source (i.e., the detected object) located in a center of the rectangular region P1 is located. A length range of the rectangular region P1 may be the axial length range of the detector array.

Referring to a in FIG. 6, for a detector A, an entire first projection region P2 (i.e., the detecting region of the detector array for the detected object) may be sieged by the detector A as an endpoint and two first siege lines led out from the detector A as side lines, to obtain a first siege region P3. Then, the first siege region P3 may be taken as the first effective detecting range in the axial view for the detector array. An end of each of the two first siege lines may be located at the endpoint at which the detector A is located (i.e., the detector A may be a local-end detector), the other end of each of the two first siege lines may be located at a position at which a detector A1 or a detector A2 is located in the axial length range of the detector array (i.e., the detector A1 or the detector A2 may be the peer-end detector), and a distance between the two first siege lines and a contour edge of the first projection region P2 may be a preset safe distance, so that the first siege region P3 may siege the entire first projection region P2.

Similarly, referring to b in FIG. 6, for a detector B, an entire first projection region P2 may be sieged by the detector B as an endpoint and two first siege lines led out from the detector B as side lines, to obtain the first siege region P3. Then, the first siege region P3 may be taken as the first effective detecting range in the axial view for the detector array. An end of each of the two first siege lines may be located at the endpoint at which the detector B is located (i.e., the detector B may be a local-end detector), the other end of each of the two first siege lines may be located at a position at which a detector B1 or a detector B2 is located in the axial length range of the detector array (i.e., the detector B1 or the detector B2 may be the peer-end detector), and a first siege line at which the detector B and the detector B1 are located may be intersected with the first projection region P2 since the detector B1 is located at an end position of the axial length range of the detector array. In addition, since the detector B2 is located at a middle position in the axial length range of the detector array, a distance between the first siege line at which the detector B and the detector B2 are located and a contour edge of the first projection region P2 may be a preset safe distance, so that the first siege region P3 may siege a part of the first projection region P2. The preset safe distance may be a minimum distance between the first siege line at which the detector B and the detector B2 are located and the contour edge of the first projection region P2 when it facilitates that the first siege region P3 sieges the part of the first projection region P2.

In another embodiment, for the circumferential view, the server may determine a second effective detecting range of each detector in the circumferential view based on the second projection region. The final detecting range in three dimensions may include the first effective detecting range and the second effective detecting range.

Specifically, the server may first siege the second projection region by using a detector as an endpoint and two corresponding second siege lines as sidelines, to obtain the second siege region. Then, a view range corresponding to the second siege region may be taken as the second effective detecting range.

An end of each of the second siege lines may be located at the endpoint, the other end of each of the second siege lines may be located within a circumferential length range of the detector array, and the second siege region is configured to siege the entire second projection region.

In an exemplary embodiment, referring to FIG. 7, FIG. 7 is a schematic diagram of an effective detecting range of a detector in a circumferential view in an embodiment of the present disclosure. Referring to a and b in FIG. 7, a circular region S1 may be a display region of the detector array in the circumferential view, and each detector in the detector array may be arranged at an edge position of the circular region S1, so as to perform signal detection in a second projection region S2 at which a radiation source (i.e., the detected object) located in a center of the circular region S1 is located. A circumference range of the circular region S1 may be the circumferential length range of the detector array.

Referring to a in FIG. 7, for a detector A, an entire second projection region S2 (i.e., the detecting region of the detector array for the detected object) may be siege by the detector A as an endpoint and two first siege lines led out from the detector A as side lines, to obtain a second siege region S3. Then, the second siege region S3 may be taken as the second effective detecting range in the circumferential view for the detector array. An end of each of the two second siege lines may be located at the endpoint at which the detector A is located (i.e., the detector A may be a local-end detector), the other end of each of the two second siege lines may be located at a position at which a detector A1 or a detector A2 is located in the circumferential length range of the detector array (i.e., the detector A1 or the detector A2 may be the peer-end detector), and a distance between the two second siege lines and a contour edge of the second projection region S2 may be a preset safe distance, so that the second siege region S3 may enclose all the second projection region S2.

Similarly, referring to b in FIG. 7, for a detector B, an entire second projection region S2 may be sieged by the detector B as an endpoint and two second siege lines led out from the detector B as side lines, to obtain the second siege region S3. Then, the second siege region S3 may be taken as the second effective detecting range in the circumferential view for the detector array. An end of each of the two second siege lines is at the endpoint at which the detector B is located (i.e., the detector B may be a local-end detector), and the other end of each of the two first siege lines may be located at a position at which a detector B1 or a detector B2 is located in the circumferential length range of the detector array (i.e., the detector B1 or the detector B2 may be the peer-end detector). A distance between each of the two second siege lines and a contour edge of the second projection region may be a preset safe distance, so that the second siege region S3 may siege the entire second projection regions S2.

In this way, in one aspect, the detection scenario of the detector array for the detected object is determined by the data feature of the detected signal data set, and then the effective detecting range of each detector is determined according to the corresponding detection scenario, so as to reconstruct the medical detecting image based on the target signal data in the corresponding effective detecting range. Therefore, a procedure of reconstructing the medical image is optimized, and compared with a manner in the related art, reconstruction efficiency of the medical image is effectively improved by a standardized execution program, and human and material power consumption is reduced. In another aspect, the trace signal emitted by the detected object is first detected by sieging the detected object in three dimensions, and then the target signal data in the effective detecting range corresponding to each detector is extracted from the trace signal according to the detecting scenario of the detector array, so as to construct the medical detecting image. Therefore, accuracy and feasibility of reconstructing the medical image are improved, and it facilitates providing further effective reference information for subsequent medical treatment.

It should be understood that, although steps in the flowcharts of FIG. 2 to FIG. 7 are sequentially displayed according to an instruction of an arrow, these steps are not necessarily sequentially performed according to an instruction of an arrow. Unless expressly stated in the specification, these steps are not performed in a strict order, and these steps may be performed in another order. In addition, at least a part of steps in FIG. 2 to FIG. 7 may include multiple steps or phases. These steps or phases are not necessarily performed at the same moment, but may be performed at different moments. An execution sequence of these steps or phases is not necessarily performed sequentially, but may be performed alternately or alternately with another step or at least a part of steps or phases in another step.

It may be understood that a same/similar part of the foregoing method embodiments in the specification may refer to each other, and each embodiment focuses on a difference from another embodiment. For related parts, referring to descriptions of another method embodiment.

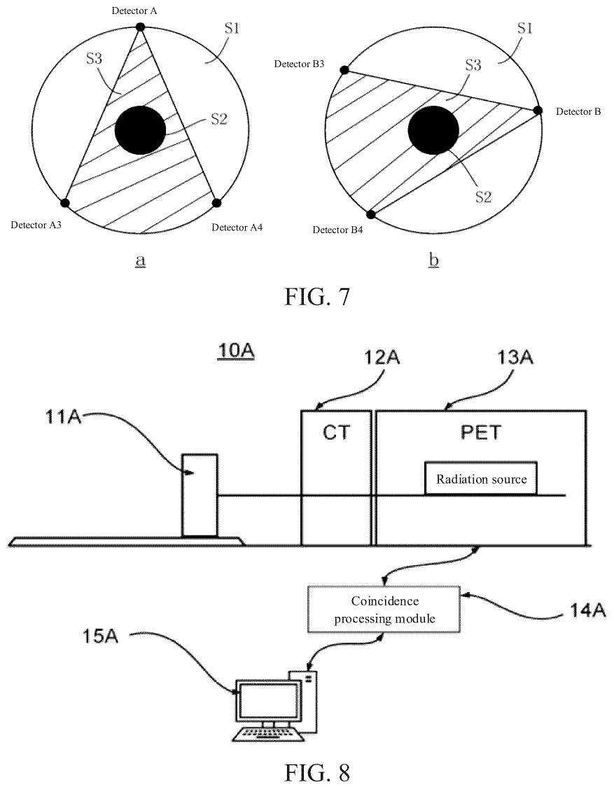

FIG. 8 is a block diagram of a PET/CT system in an embodiment of the present disclosure. Referring to FIG. 8, the PET/CT system 10A may include a scanning bed 11A, a CT device 12A, a PET device 13A, a coincidence processing module 14A, and a display device 15A.

The scanning bed 11A is configured to support the detected object (i.e., the radiation source), and position the detected object in a required position in the PET device 13A. In some embodiments, the detected object may lie on the scanning bed 11A. The scanning bed 11A may move under the control of the server and reach a desired location of the PET device 13A. In some embodiments, the scanning bed 11A may have a relatively long axial view and a relatively large circumferential view, such as an axial view of 2 meters.

The CT device 12A is configured to scan CT data of the detected object to reconstruct a CT image. The CT image may provide anatomical structure information with high resolution, including detailed structures of a bone, an organ, and a soft tissue. This information may help the physician to accurately locate the specific location of the abnormal metabolic region in the PET image, thereby more accurately diagnosing the lesion.

The PET device 13A is configured to perform signal detection on the detected object by the detector array to obtain the signal data set. The detector array is configured to siege the detected object in three dimensions and detect the trace signal emitted by the detected object to obtain the signal data set.

The coincidence processing module 14A is configured to perform a coincidence operation on the target signal data of the detector in the respective effective detecting range, so as to reconstruct a medical detecting image (i.e., a PET image) for the detected object according to an operation result of the target signal data.

The display device 15A is configured to display a reconstructed CT image and a reconstructed PET image on a display interface, so that the physician and the researcher perform analysis and diagnosis.

In a specific implementation scenario, the PET/CT system 10A may first collect statistics about the counting rate of the positron signal on the signal data set collected by the PET device 13A in real time, so as to determine the current scanning protocol of the PET device 13A. Then, an ineffective scanning view region of the PET device 13A may be determined according to the current scanning protocol of the PET device 13A (i.e., a non-concern region in the coincidence lines may be determined). Then, it is determined, according to the ineffective scanning view region, that a coincidence line that needs to be closed in the PET device 13A (i.e., it is determined that the response line in the coincidence lines which outside a limiting coincidence range or an effective view about the detector crystal). Finally, the target signal data of the unclosed coincidence line may be extracted from the signal data set, and the PET image of the detected object may be reconstructed based on the target signal data.

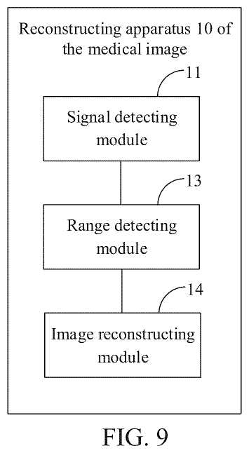

FIG. 9 is a block diagram of a reconstructing apparatus of a medical image in an embodiment of the present disclosure. Referring to FIG. 9, the reconstructing apparatus 10 of the medical image includes a signal detecting module 11, a range detecting module 13, and an image reconstructing module 14.

The signal detecting module 11 is configured for acquiring a signal data set obtained by performing signal detection on a detected object via a detector array. The detector array is configured to siege the detected object in three dimensions and detect a trace signal emitted by the detected object to obtain the signal data set.

The range detecting module 13 is configured for determining an effective detecting range of each detector in the detector array when a detecting scenario is a target scenario. Effective detecting ranges corresponding to the detector in different detection scenarios are different.

The image constructing module 14 is configured for extracting target signal data of each detector within a respective effective detecting range from the signal data set, and reconstructing a medical detecting image of the detected object based on the target signal data.

In some embodiments, the range detecting module 13 is further configured for acquiring a detecting region of the detector array for the detected object, and determining, based on the detecting region, the effective detecting range corresponding to each detector.

In some embodiments, the detecting region may include a first projection region of the detected object in an axial view of the detector array. The range detecting module 13 is further configured for determining, based on the first projection region, a first effective detecting range of each detector in the axial view.

In some embodiments, for the axial view, the range detecting module 13 is further configured for taking the detector as an endpoint and two corresponding first siege lines as sidelines, sieging the first projection region to obtain a first siege region, and taking a view range corresponding to the first siege region as the first effective detecting range. An end of each of the first siege lines may be located at the endpoint, the other end of each of the first siege lines may be located within an axial length range of the detector array, and the first siege region is configured to siege at least a part of the first projection region.

In some embodiments, the detecting region may include a second projection region of the detected object in a circumferential view of the detector array. The range detecting module 13 is further configured for determining, based on the second projection region, a second effective detecting range of each detector in the circumferential view.

In some embodiments, for the circumferential view, the range detecting module 13 is further configured for taking the detector as an endpoint and two corresponding second siege lines as sidelines, sieging the second projection region to obtain a second siege region, and taking a view range corresponding to the second siege region as the second effective detecting range. An end of each of the second siege lines may be located at the endpoint, the other end of each of the second siege lines may be located within a circumferential length range of the detector array, and the second siege region is configured to siege all the second projection region.

In some embodiments, each detector has at least one coincidence line within the respective effective detecting range, an end of the at least one coincidence line is a local-end detector corresponding to the effective detecting range, and the other end of the at least one coincidence line is a peer-end detector. The image constructing module 14 is further configured for extracting, from the signal data set, target trace signals of the local-end detector and the peer-end detector those are on each of the at least one coincidence line, and taking the target trace signals as the target signal data. Each of the at least one coincidence line may belong to the corresponding effective detecting range of each detector.

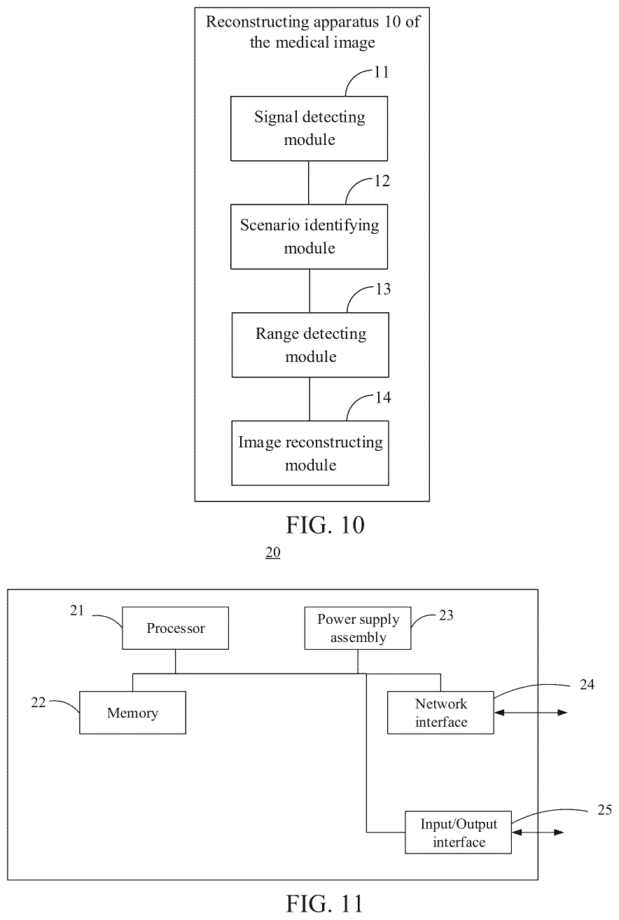

Referring to FIG. 10, the reconstructing apparatus 10 of the medical image may further include a scenario identifying module 12, which is configured for determining the detecting scenario of the detector array for the detected object based on a data feature of the signal data set.

In some embodiments, the scenario identifying module 12 is further configured for performing signal identification on each signal data in the signal data set, determining statistical data of the trace signal emitted from the detected object based on the radioactive tracer, and determining the detecting scenario of the detector array based on an activity level. The statistical data is configured to represent the activity level of the radioactive tracer in the detected object.

In some embodiments, the scenario identifying module 12 is further configured for identifying a preset radioactive signal and a positron signal that are emitted by the detected object in a coincident time window, obtaining a data amount of the radioactive signal and a counting rate of the positron signal, and taking either or both of the data amount and the counting rate as the statistical data for the trace signal.

FIG. 11 is a block diagram of a computer device 20 in an embodiment of the present disclosure. For example, the computer device 20 may be an electronic device, an electronic component, a server array, or the like. Referring to FIG. 11, the computer device 20 may include a processor 21. The processor 21 may further be a processor set, which may include one or more processors. The computer device 20 may include a memory resource represented by a memory 22. The memory 22 may store a computer program, such as an application program. The computer program stored in the memory 22 may include one or more modules corresponding to a group of executable instructions. In addition, the processor 21 is configured to implement the foregoing reconstructing method of the medical image when the computer program is executed.

In some embodiments, the computer device 20 may be an electronic device, and a computing system in the electronic device may run one or more operating systems, including any operating system discussed above and any commercially available server operating system. The computer device 20 may further run any one of various additional server applications and/or intermediate layer applications, including an HTTP (Hypertext Transfer Protocol) server, an FTP (File Transfer Protocol) server, a CGI (Common Gateway Interface) server, a super server, a database server, or the like. Exemplary database servers may include, but are not limited to, database servers available from (international commercial machines) or the like.

In some embodiments, the processor 21 may generally control the overall operation of the computer device 20, such as operations associated with display, data processing, data communication, and recording operations. The processor 21 may include one or more processor components to execute the computer program, so as to complete all or a part of the steps of the foregoing method. In addition, the processor component may include one or more modules, so as to facilitate interaction between the processor component and another component. For example, the processor component may include a multimedia module, so as to facilitate use of the multimedia component to control interaction between the computer device 20 of the user and the processor 21.

In some embodiments, the processor component in the processor 21 may further be referred to as a CPU (Central Processing Unit). The processor component may be an electronic chip with a signal processing capability. The processor may further be a general-purpose processor, a digital signal processor (DSP), an application specific integrated circuit (ASIC), a field programmable gate array (FPGA) or another programmable logic device, a discrete gate or transistor logic device, or a discrete hardware component. The general-purpose processor may be a microprocessor, or any conventional processor component. In addition, the processor component may be jointly implemented by an integrated circuit chip.

In some embodiments, the memory 22 is configured to store various types of data to support operation at the computer device 20. Examples of these data may include instructions, data collection, messages, pictures, videos, or the like for any application program or method to operate on the computer device 20. The memory 22 may be implemented by any type of volatile or non-volatile storage device or a combination thereof, such as a static random access memory (SRAM), an electrically erasable programmable read-only memory (EEPROM), an erasable programmable read-only memory (EPROM), a programmable read-only memory (PROM), a read-only memory (ROM), a magnetic memory, a flash memory, a magnetic disk, an optical disc, or a graphene memory.

In some embodiments, the memory 22 may be a memory bar, a TF (Trans-flash) card, or the like, and all information in the computer device 20, including input raw data, a computer program, an intermediate running result, and a final running result, may be stored in the memory 22. In some embodiments, the memory 22 may store and extract information according to a location specified by the processor. In some embodiments, with the memory 22, the computer device 20 may have a memory function, so that normal operation may be ensured. In some embodiments, the memory 22 of the computer device 20 may be classified into a primary memory (memory) and an auxiliary memory (external memory) by use, or may be classified into an external memory and an internal memory. The external memory may be usually a magnetic medium or an optical disc, and may store information for a long time. The memory may refer to the storage member on the main board, which is configured to store the data and programs currently being executed, but is only configured to temporarily store the programs and data, and the data will be lost when power off.

In some embodiments, the computer device 20 may further include: a power supply assembly 23 configured to perform power management of the computer device 20, a wired or wireless network interface 24 configured to connect the computer device 20 to a network, and an input/output (I/O) interface 25. The computer device 20 may operate an operating system that is stored in the memory 22, such as Windows Server, Mac OS X, Unix, Linux, FreeBSD, or the like.

In some embodiments, the power supply assembly 23 may provide power for various components of the computer device 20. The power supply assembly 23 may include a power supply management system, one or more power supplies, and other components associated with generating, managing, and allocating power for the computer device 20.

In some embodiments, the wired or wireless network interface 24 is configured to facilitate wired or wireless communication between the computer device 20 and another device. The computer device 20 may access a wireless network based on a communications standard, such as WIFI, an operator network (such as 2G, 3G, 4G, or 5G), or a combination thereof.

In some embodiments, the wired or wireless network interface 24 may receive broadcast signals or broadcast-related information from an external broadcast management system via a broadcast channel. In an exemplary embodiment, the wired or wireless network interface 24 may further include a near field communication (NFC) module to facilitate short-range communication. For example, the NFC module may be implemented based on a radio frequency identification (RFID) technology, an infrared data association (IrDA) technology, an ultra-wideband (UWB) technology, a Bluetooth (BT) technology, and another technology.

In some embodiments, the input/output (I/O) interface 25 may provide an interface between the processor 21 and a peripheral interface module. The peripheral interface module may be a keyboard, a tap wheel, a button, or the like. The button may include but is not limited to a home button, a volume button, a start button, and a lock button.

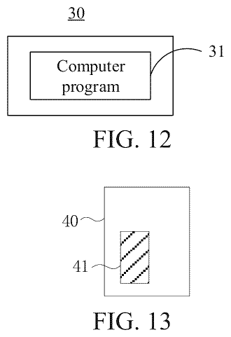

FIG. 12 is a block diagram of a computer-readable storage medium 30 in an embodiment of the present disclosure. The computer-readable storage medium 30 may store a computer program 31, and the computer program 31 may implement the foregoing reconstructing method for the medical image when being executed by a processor.

When units integrated into the functional units in the embodiments of the present

disclosure are implemented in a form of a software functional unit and sold or used as an independent product, the units may be stored in the computer-readable storage medium 30. Based on this understanding, the essential technical solutions of the present disclosure, a part contributing to the related art, or all or a part of the technical solutions may be implemented in a form of a software product. The computer program 31 in the computer-readable storage medium 30 may include several instructions for instructing a computer device (which may be a personal computer, a system server, or a network device), an electronic device (such as an MP3 or a MP4, an intelligent terminal such as a mobile phone, a tablet computer, or a wearable device, or a desktop computer), or a processor to perform all or a part of the steps of the methods of the embodiments of the present disclosure.

FIG. 13 is a block diagram of a computer program product 40 in an embodiment of the present disclosure. The computer program product 40 may include a program instruction 41, and the program instruction 41 may be executed by a processor of the server 20 to implement the foregoing reconstructing method of the medical image.

One skilled in the art should understand that the embodiments of the present disclosure may provide the reconstructing method of the medical image, the reconstructing apparatus 10 of the medical image, the computer device 20, the computer-readable storage medium 30, or the computer program product 40. Therefore, the present disclosure may use a form of a complete hardware embodiment, a complete software embodiment, or an embodiment combined with software and hardware aspects. In addition, the present disclosure may be used in a form of a computer program product 40 implemented on one or more computer program instructions 41 (including but not limited to a disk memory, a CD-ROM, and an optical memory) that include computer available program code.

The present disclosure is described with reference to a flowchart and/or a block diagram of the reconstructing method of the medical image, the reconstructing apparatus 10 of the medical image, the computer device 20, the computer-readable storage medium 30, or the computer program product 40 according to embodiments of the present disclosure. It should be understood that the computer program product 40 may implement each process and/or block in the flowchart and/or the block diagram, and a combination of processes and/or blocks in the flowchart and/or the block diagram. The computer program product 40 may be provided to a processor of a general-purpose computer, a dedicated computer, an embedded processor, or another programmable data processing device to produce a machine, such that the program instructions 41 executed by a processor of a computer or another programmable data processing device may generate an apparatus for implementing a function specified in one or more processes of the flowchart and/or one or more blocks of the block diagram.

These computer program products 40 may also be stored in a computer-readable memory that can instruct a computer or another programmable data processing device to operate in a specific manner, so that the program instructions 41 stored in the computer program product 40 may generate a manufacturer including an instruction apparatus. The instruction apparatus may implement a function specified in one or more procedures of the flowchart and/or one or more blocks of the block diagram.

These program instructions 41 may also be loaded onto a computer or another programmable data processing device, so that a series of operation steps may be performed on the computer or another programmable device to generate computer-implemented processing, so that the program instructions 41 executed on the computer or another programmable device may provide steps for implementing a function specified in one or more processes of the flowchart and/or one or more blocks of the block diagram.

It should be noted that the foregoing various methods, apparatuses, electronic devices, computer-readable storage media, computer program products, and the like may further include another implementation manner according to descriptions of method embodiments. For a specific implementation manner, reference may be made to descriptions of related method embodiments, and details are not described herein again.

One skilled in the art may easily think of another implementation solution of the present disclosure after considering the specification and the invention disclosed herein. The present disclosure is intended to cover any variations, uses, or adaptations of the present disclosure that follow the general principles of the present disclosure and include well-known or commonly used technical means in the art that are not disclosed herein. The specification and embodiments are merely exemplary, and the true scope and spirit of the present disclosure are stated by the claims.

It should be understood that the present disclosure is not limited to the precise structure described above and shown in the accompanying drawings, and various modifications and changes may be made without departing from its scope. The scope of the present disclosure is limited only by the appended claims.

What is claimed is:

1. A reconstructing method of a medical image, comprising:
acquiring a signal data set obtained by performing signal detection on a detected object via a detector array, wherein the detector array is configured to siege the detected object in three dimensions and detect a trace signal emitted by the detected object to obtain the signal data set;
determining an effective detecting range of each detector in the detector array when a detecting scenario is a target scenario; and
extracting target signal data of each detector within a respective effective detecting range from the signal data set, and
reconstructing a medical detecting image of the detected object based on the target signal data.

2. The method of claim 1, wherein determining the effective detecting range of each detector in the detector array further comprises:
acquiring a detecting region of the detector array for the detected object; and
determining, based on the detecting region, the effective detecting range corresponding to each detector.

3. The method of claim 2, wherein the detecting region comprises a first projection region of the detected object in an axial view of the detector array; and
determining, based on the detecting region, the effective detecting range corresponding to each detector further comprises:
determining, based on the first projection region, a first effective detecting range of each detector in the axial view.

4. The method of claim 3, wherein determining the effective detecting range corresponding to each detector further comprises:
for the axial view, taking the detector as an endpoint and two corresponding first siege lines as sidelines, sieging the first projection region to obtain a first siege region, and taking a view range corresponding to the first siege region as the first effective detecting range,
wherein an end of each of the first siege lines is located at the endpoint, the other end of each of the first siege lines is located within an axial length range of the detector array, and the first siege region is configured to siege at least a part of the first projection region.

5. The method of claim 2, wherein the detecting region comprises a second projection region of the detected object in a circumferential view of the detector array; and
determining, based on the detecting region, the effective detecting range corresponding to each detector further comprises:
determining, based on the second projection region, a second effective detecting range of each detector in the circumferential view.

6. The method of claim 5, wherein determining the effective detecting range corresponding to each detector further comprises:
for the circumferential view, taking the detector as an endpoint and two corresponding second siege lines as sidelines, sieging the second projection region to obtain a second siege region, and taking a view range corresponding to the second siege region as the second effective detecting range,
wherein an end of each of the second siege lines is located at the endpoint, the other end of each of the second siege lines is located within a circumferential length range of the detector array, and the second siege region is configured to siege all the second projection region.

7. The method of claim 1, wherein each detector has at least one coincidence line within the respective effective detecting range, an end of the at least one coincidence line is a local-end detector corresponding to the effective detecting range, and the other end of the at least one coincidence line is a peer-end detector; and
extracting target signal data of each detector within the respective effective detecting range from the signal data set further comprises:
extracting, from the signal data set, target trace signals of the local-end detector and the peer-end detector those are on each of the at least one coincidence line, and taking the target trace signals as the target signal data, wherein each of the at least one coincidence line belongs to the corresponding effective detecting range of each detector.

8. The method of claim 1, further comprising: determining the detecting scenario of the detector array for the detected object based on a data feature of the signal data set.

9. The method of claim 8, wherein determining the detecting scenario of the detector array for the detected object based on the data feature of the signal data set further comprises:
performing signal identification on each signal data in the signal data set, and determining statistical data of the trace signal sent from the detected object based on the radioactive tracer, wherein the statistical data is configured to represent an activity level of the radioactive tracer in the detected object; and
determining the detecting scenario of the detector array based on the activity level.

10. The method of claim 9, wherein performing signal identification on each signal data in the signal data set, and determining statistical data of the trace signal sent from the detected object based on the radioactive tracer further comprises:
identifying a radioactive signal and a positron signal that are emitted by the detected object in a coincident time window, obtaining a data amount of the preset radioactive signal and a counting rate of the positron signal, and taking either or both of the data amount and the counting rate as the statistical data for the trace signal.

11. A reconstructing apparatus of a medical image, comprising:
means for acquiring a signal data set obtained by performing signal detection on a detected object via a detector array, wherein the detector array is configured to siege the detected object in three dimensions and detect a trace signal emitted by the detected object to obtain the signal data set;
means for determining an effective detecting range of each detector in the detector array when the detecting scenario is a target scenario; and
means for extracting target signal data of each detector within a respective effective detecting range from the signal data set, and reconstructing a medical detecting image of the detected object based on the target signal data.

12. A computer device, comprising a memory and a processor, wherein the memory stores a computer program, which is executed by a processor to implement steps of the method of claim 1.

13. The computer device of claim 12, determining the effective detecting range of each detector in the detector array further comprises:
acquiring a detecting region of the detector array for the detected object; and
determining, based on the detecting region, the effective detecting range corresponding to each detector.

14. The computer device of claim 13, wherein the detecting region comprises a first projection region of the detected object in an axial view of the detector array; and
determining, based on the detecting region, the effective detecting range corresponding to each detector further comprises:
determining, based on the first projection region, a first effective detecting range of each detector in the axial view.

15. The computer device of claim 14, wherein determining the effective detecting range corresponding to each detector further comprises:
for the axial view, taking the detector as an endpoint and two corresponding first siege lines as sidelines, sieging the first projection region to obtain a first siege region, and taking a view range corresponding to the first siege region as the first effective detecting range,
wherein an end of each of the first siege lines is located at the endpoint, the other end of each of the first siege lines is located within an axial length range of the detector array, and the first siege region is configured to siege at least a part of the first projection region.

16. The computer device of claim 13, wherein the detecting region comprises a second projection region of the detected object in a circumferential view of the detector array; and
determining, based on the detecting region, the effective detecting range corresponding to each detector further comprises:
determining, based on the second projection region, a second effective detecting range of each detector in the circumferential view.

17. The computer device of claim 16, wherein determining the effective detecting range corresponding to each detector further comprises:
for the circumferential view, taking the detector as an endpoint and two corresponding second siege lines as sidelines, sieging the second projection region to obtain a second siege region, and taking a view range corresponding to the second siege region as the second effective detecting range,
wherein an end of each of the second siege lines is located at the endpoint, the other end of each of the second siege lines is located within a circumferential length range of the detector array, and the second siege region is configured to siege all the second projection region.

18. The computer device of claim 12, wherein each detector has at least one coincidence line within the respective effective detecting range, an end of the at least one coincidence line is a local-end detector corresponding to the effective detecting range, and the other end of the at least one coincidence line is a peer-end detector; and
extracting target signal data of each detector within the respective effective detecting range from the signal data set further comprises:
extracting, from the signal data set, target trace signals of the local-end detector and the peer- end detector those are on each of the at least one coincidence line, and taking the target trace signals as the target signal data, wherein each of the at least one coincidence line belongs to the corresponding effective detecting range of each detector.

19. The computer device of claim 12, wherein the computer program is executed by the processor to implement the following step: determining the detecting scenario of the detector array for the detected object based on the data feature of the signal data set.

20. A computer-readable storage medium, on which a computer program is stored, wherein the computer program is executed by a processor to implement steps of the method of claim 1.
